(19) United States
(12) Patent Application Publication
(10) Pub. No.: US 2007/0093709 A1
(43) Pub. Date: Apr. 26, 2007

(54) Surgical navigation markers

(76) Inventor: Dennis Lee Abernathie, Columbia, MO (US)

Correspondence Address: R. Scott Kimsey;Sonnenschein Nath & Rosenthal LLP, Sears Tower, Wacker Drive Station, P.O. Box #061080, Chicago, IL, 60606-1080, US

(21) Appl. No.: 11/258,697

(22) Filed: Oct. 26, 2005

(51) Int. Cl. A61B 5/05

(52) U.S. Cl. 600/407; 600/424

(57) ABSTRACT

The present invention provides a surgical navigation marker having a shaft with a head at one end and a point at the other. The point is adapted for easy insertion of the navigation marker in a portion of a patient's anatomy, such as bone. The head of the navigation marker has a depression formed therein that is sized and shaped for precise registration by an imaging or tracking system.

### CROSS-REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### BACKGROUND OF THE INVENTION

The present invention relates generally to a surgical navigation device and method, and more specifically to a device and method for providing accurate reference points for surgical imaging.

The use of imaging methods during surgical procedures is well-known, and a variety of methods and devices exist related to such procedures. The current art ranges from relatively simple to relatively complex methods and instrumentation, yet each of these methods and devices is directed generally to the same goal: accurate registration of patient anatomy and surgical instrumentation during a surgical procedure.

Registration refers generally to a cross-correlation of various components in space. The components that must be cross-correlated are varied, and may include patient anatomy, surgical devices such as a drill, scalpel, or ablator, and various images such as pre-operative CT or MRI images, or intraoperative x-ray, ultrasound, or other images. Many known methods of surgical navigation require both pre-operative and intra-operative steps. For example, various markers may be placed on a patient's body pre-operatively, on or near the surgical site, and an image that includes these markers may be obtained via pre-operative MRI, CT, or other imaging methods. Computer technology can then be used to generate a three-dimensional (3D) model from which a surgical plan can be developed. This model may also include the markers placed on the patient's body prior to imaging. The surgical procedure may then be performed according to the plan based on the pre-operative images and the position of the markers in those images. Such a method benefits from the fact that detailed, high-resolution images may be used to image the patient's anatomy and develop surgical models. This method has drawbacks, however, in that there is no real-time image feedback, so the movement of targets (such as surgical instruments) relative to the markers or patient's anatomy cannot be tracked during the surgical procedure.

Some of the limitations of the procedure above can be addressed by providing additional images during the surgical procedure, preferably with real-time feedback during surgery. During the surgical procedure, the per-operative image may be superimposed on an x-ray, ultrasound, fluoroscopic or other image acquired during the procedure. The markers placed on the patient's body and captured in the pre-operative image may serve as reference points for the superimposition of the two images. Further, the markers may be contacted by surgical instrumentation, thus enabling the markers to serve as a starting reference point for displaying the position of surgical instruments during the surgical procedure. This provides for real-time registration of surgical tools and patient anatomy.

Each of the above methods, and numerous other methods available, rely on proper registration, most commonly via the use of landmarks such as natural markers or man-made fiducial markers. Natural landmarks, such as bone structures or other portions of the patient's anatomy may be used. In some instances, a combination of natural landmarks and man-made fiducial markers may be used.

Regardless of the type of marker used, a good marker has a number of characteristics that lend it to use in a surgical procedure. A good marker is capable of rigid attachment to the patient, instrument, or other locality sought to be registered during the surgical procedure. Further, a good marker is easily seen and recognized by the medical imaging software used during a surgical procedure, as well as by any tracking device used to track the movements of the marker during a procedure. Preferably, a surgical marker is relatively small and minimally invasive.

Currently used surgical navigation markers suffer from various drawbacks. With respect to natural landmarks, for example, the portion of the patient's anatomy used is generally curved or irregular in shape, making it difficult for imaging and tracking software to reference a single point on the landmark. As a result, there is error in the images generated during surgery in that the position of surgical instruments, patient anatomy, and other reference points may be different than shown in the image. Man-made fiducial markers may be more reliable, but even these devices suffer from error. As a rule, the smaller the navigation marker, the more precisely the imaging software is able to register the location of the marker. This is due to the fact that the imaging and tracking software is focusing on a smaller area (more akin to a point), rather than on a large surface. Thus, the deviation from one aligning or focusing event to the next is reduced. As the size of the marker is reduced, however, the marker becomes harder to use, both because it is harder for the surgeon to manipulate, and because it is harder to see and may inadvertently be lost or left inside of the patient following the surgical procedure.

What is needed, therefore, is a surgical navigation marker that is readily fixedly attached to a point to be tracked, large enough to be easily seen and manipulated, but structure such that the imaging and tracking software can easily fix on a specific point on the marker, thereby reducing error in registration among the various markers. What is further needed is a navigation marker that contains identification information specific to that marker, and a method of utilizing the same.

### BRIEF SUMMARY OF THE INVENTION

The present invention provides a surgical navigation marker having a shaft with a head at one end and a point at the other. The point is adapted for easy insertion of the navigation marker in a portion of a patient's anatomy. The head of the navigation marker has a depression formed therein, which is sized and shaped for precise registration by an imaging or tracking system.

In another aspect of the present invention, the surgical navigation marker further includes an opening extending from a surface of the depression into the body of the surgical navigation marker. This opening is capable of registration by an imaging or tracking system.

In a further aspect of the present invention, a surgical navigation marker is provided having a threaded shaft.

In still another aspect of the present invention, a surgical marker is provided having at least one flattened portion on a head thereof to allow easy manipulation of the surgical navigation marker.

In another aspect of the present invention, at least a portion of the depression formed in the surgical navigation marker is provided with a color, with scoring, or with texturing that is capable of registration by an imaging or tracking system.

In another aspect of the present invention, an inversion is provided extending outwardly from the depression formed in the surgical navigation marker, the inversion being capable of registration by an imaging or tracking system. The inversion may be colored, scored, or textured in such a way as to facilitate registration by an imaging or tracking system.

In still another aspect of the present invention, a surgical navigation marker according to the present invention may be provided with a passive or active RFID portion.

In another aspect of the present invention, a surgical navigation marker according to the present invention may be attached to a surgical instrument for registration of the position of the surgical instrument by an imaging or tracking system.

In another aspect of the present invention, a method of defining a sterile field during a surgical procedure is provided. The method includes providing at least three surgical navigation markers in accordance with the present invention, positioning each of the surgical navigation markers along the edge of a sterile field, providing an imaging system, and registering the position of each of the surgical navigation markers using the imaging system. The imaging system may also display the boundary of the sterile field as determined by the positions of the surgical navigation markers.

In still another aspect of the present invention, a method of maintaining a sterile field is provided. The method includes providing a plurality of surgical navigation markers having passive RFID portions associated therewith, positioning the plurality of surgical navigation markers to define the border of a sterile field, providing at least one surgical instrument with an active RFID portion such that the active RFID portion of the surgical instrument is capable of energizing the passive RFID portion of the surgical markers if the active RFID portion of the surgical instrument comes within a predetermined distance of the passive RFID portion of the surgical navigation marker, and providing an alarm to alert a user when a surgical navigation marker is within a predetermined distance of a surgical navigation marker.

In another aspect of the present invention, a method of establishing a Cartesian plane within a surgical field is provided. The method includes utilizing the relative position of each of a plurality of surgical navigation markers to establish the Cartesian plane.

### BRIEF DESCRIPTION OF THE DRAWINGS

FIG. 1 is a perspective view of a navigation marker constructed in accordance with the teachings of the present invention.

FIG. 2 is a cross-sectional view of the navigation marker of FIG. 1 taken along line 2-2.

FIG. 3 is a cross-sectional view of an alternative embodiment of the navigation marker of FIG. 1.

FIG. 4 is an elevational view of a navigation marker of the present invention attached to a patient's bone during a surgical procedure.

### DETAILED DESCRIPTION OF THE INVENTION

As used herein, the terms “navigation marker,” “marker,” and “surgical navigation marker” are used interchangeably, unless otherwise indicated, to refer to a construction used in a surgical procedure for registering points on a patient's anatomy or on surgical instruments or other portions of the operative field.

As used herein, the terms “imaging software,” “tracking software,” “imaging system,” and “tracking system” are all used to refer generally to a software or other system designed to register navigation markers, patient anatomy, surgical instrumentation, and other portions of the surgical field both before and during a surgical procedure.

Turning now to the drawings, wherein like numerals indicate like parts, the numeral 10 refers generally to a navigation marker constructed in accordance with the teachings of the present invention.

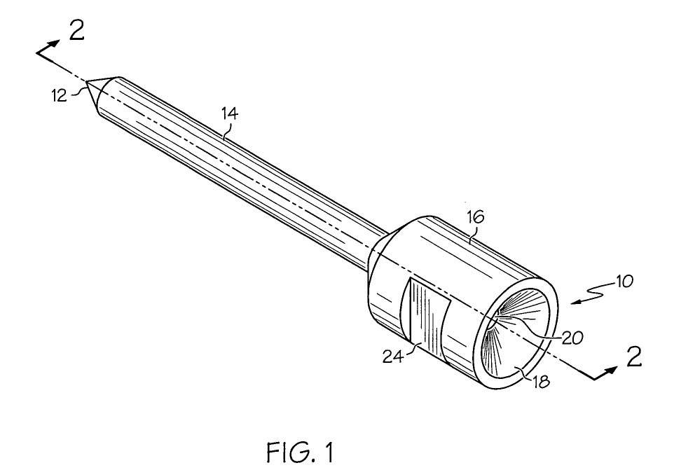

In the embodiment of the present invention shown in FIG. 1, navigation marker 10 is presented in the form of a pin that may be inserted into a bone of a patient undergoing a surgical procedure. Navigation marker 10 includes a sharpened end 12, which allows for easy insertion of navigation marker 10 into the bone or other area of a patient's body, a shaft portion 14, and a head portion 16 disposed at the opposite end of shaft portion 14 from end 12. Navigation marker 10 also includes a symmetrical depression 18 to which a navigation system can be focused, and a flattened portion 24 provided for ease of manipulation of marker 10. Flattened portion 24 can be gripped, for example, by a tool or even by a user's fingers. In the embodiment of navigation marker 10 shown in FIG. 1, all or a portion of shaft 14 may be threaded such that navigation marker 10 is rotatably insertable into bone or portions of a patient's anatomy for use during a surgical procedure.

Depression 18 of navigation marker 10 provides the mechanism by which the imaging or tracking system used in a surgical procedure is able to maintain accurate registration of one or more such markers. In the embodiment of navigation marker 10 shown in FIG. 1, depression 18 is a concave depression that tapers to a small opening 20 in head 16 of the marker. In alternative embodiments of marker 10, opening 20 may be eliminated, allowing depression 18 to simply taper to a relatively small area at the bottom of the depression, defining a point. Whether an opening 20 is present, or a point is defined by depression 18 itself, it is not necessary that the bottom of depression 18 have any particular size or shape, but only that the imaging or tracking system used recognize the bottom of depression 18 as a ‘point’ with which registration of navigation marker 10 can be associated.

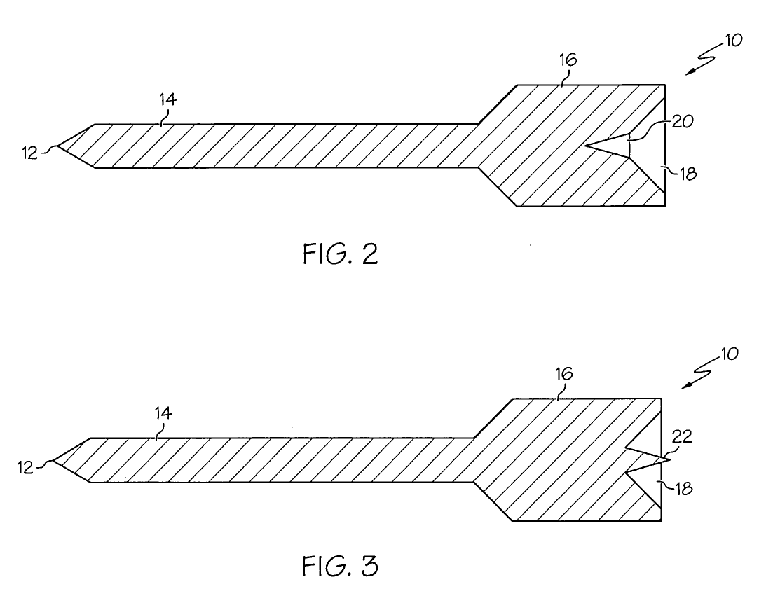

FIG. 2 provides a cross-sectional view of navigation marker 10 along line 2-2. As shown, depression 18 is generally annular, with a diameter that becomes progressively small until reaching opening 20. Although it is preferred that opening 20 or a point defined by depression 18 be defined simply by the size and/or shape of the opening 20, or the countours of depression 18 itself, it is contemplated that opening 20 (or any ‘point’ defined by depression 18 as it becomes progressively smaller) may be further distinguished from the rest of navigation marker 10 by color, or by scoring or texturing of the surface of navigation marker 10 at or near the location of opening 20 or the point defined by depression 18. Such measures may increase the reliability with which an imaging or tracking system is able to register a point indicated by navigation marker 10.

FIG. 3 provides a cross-sectional view of another embodiment of navigation marker 10, along a similar line as that of line 2-2 in FIG. 1. In the embodiment of navigation marker 10 shown in FIG. 3, an inversion 22 is positioned at the bottom of depression 18, such that an upwardly-jutting portion is formed. This provides a focal point for the imaging or tracking system used during a surgical procedure. As with point 20, it is preferred that inversion 22 simply be formed from the material of navigation marker 10. Inversion 22 may, however, be colored or otherwise marked, such as with scoring or texturing, such that inversion 22 is more easily recognized and tracked by the imaging or tracking system.

Though particular embodiments of the cross-sectional structure of depression 18 are shown in FIGS. 2 and 3, it is contemplated that any suitable structure may be used. Any number of modifications to the shape or size of depression 18 or opening 20 will be apparent to those of skill in the art upon reading this disclosure, and it is contemplated that such modifications are within the spirit and scope of the present invention. In addition, while depression 18 is generally annular, it is contemplated that depression 18 may be of any geometric shape or design that is susceptible to suitable registration by an imaging or tracking system.

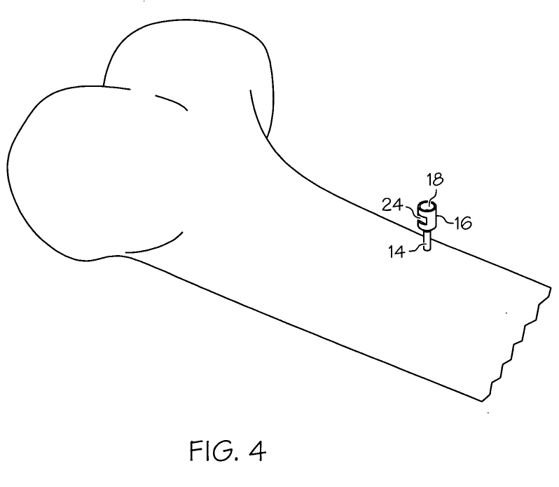

When in use, navigation marker 10 is located on patient's body, such as, for example, by insertion into bone as shown in FIG. 4. Marker 10 is placed in position prior to the initial imaging of the patient or the surgical site. Thus, the location of navigation marker 10 is established in the presurgical c-arm image. The imaging or tracking software to be used prior to and during surgery may focus on readily identifiable features of navigation marker 10, such as opening 20 or inversion 22 or depression 18 of the marker. This provides a tight focus for the imaging or tracking software, such that error in registration between various markers and other portions of the pre-operative or intra-operative image is reduced. In order to provide even better registration, the imaging or tracking system may be calibrated with a specific navigation marker, or with multiple markers, prior to use of the same markers during a surgical procedure.

In one aspect of the present invention, a surgical navigation marker constructed in accordance with the teachings of the present invention is used to verify a navigation or tracking system. For example, a navigation reference is typically attached to a C-arm as well as the patient, such that when a photograph of the operative site is taken, the C-arm is included in the photograph and the tracking system relates the position of the C-arm to that of the navigation reference attached to the patient. The distance and direction with respect to the navigation reference provide a location that is superimposed on the photograph. If the navigation reference is moved, however, registration is lost. If the loss of registration is not recognized, errors in the surgical procedure may result. A surgical navigation marker constructed in accordance with the present invention may be used to confirm a placement on the photograph relative to a known point. In this manner, registration may be verified.

Various navigation markers constructed in accordance with the teachings of the present invention may also be incorporated into surgical instruments. Such navigation markers may include those that are removably attached to such instruments prior to a surgical procedure, or markers that are embedded within surgical instruments or portions thereof such that they become, in effect, permanent parts of the surgical instrument. During a surgical procedure, and imaging or tracking system can track the position of the surgical instruments relative to patient anatomy and/or other navigation markers, fixed or otherwise.

Although the embodiments of navigation marker 10 described above are identified by the imaging or tracking software according to structural features of the navigation marker, it is contemplated that other methods of identifying and locating the markers may also be utilized. For example, Radio-Frequency Identification (RFID) technology may be used in association with navigation markers constructed accordance with teachings of the present invention. The RFID technology used may be passive or active.

Passive RFID markers generally include limited information and, as used herein, may simply contain a numerical identifier that serves to identify the specific marker to the imaging or tracking software. Such markers have no internal power supply, relying on an incoming radio-frequency signal to induce a small current within the marker. This current allows the marker to transmit a response to an imaging or tracking system being used to locate the marker. Such an embodiment of the present navigation marker may be particularly useful when multiple markers are used. The numerical identifier present in each marker may be correlated to a specific location on the patient's anatomy. In embodiments of the present navigation markers utilized within surgical instruments, a numerical identifier transmitted by a passive RFID marker may be correlated within the tracking system to a specific surgical instrument. The tracking system may then contain further information specific to that instrument.

Active RFID markers include an internal power supply of their own, and may contain more information than a passive RFID marker. A navigation marker using active RFID technology may not only transmit a numerical identifier, but may contain patient-specific information that is utilized by imaging or tracking software during a surgical procedure. In embodiments of the present navigation markers utilized within surgical instruments, an active RFID marker may include detailed information concerning the specific surgical instrument in which the marker is embedded, or to which the marker is attached. Further, in the case of both active and passive RFID navigation markers, the markers may aid in identifying and locating surgical instruments at the end of a surgical procedure so that it is apparent that all instruments are accounted for at the end of the procedure.

The present invention provides for numerous advantages when multiple navigation markers are used. One such advantage is the ability to define a coordinate system such as a Cartesian plane. Four navigation markers constructed in accordance with the teachings of the present invention can be utilized to form a Cartesian plane. Any point along that plane can serve as a Cartesian reference. By relating the plane and reference to an image provided during the surgical procedure, precise registration of a particular location or object can be achieved. Further, in the event that there is movement of the surgical navigation markers, the relationship between the markers undergoes a corresponding change, and the coordinate system can be reestablished to reflect that change. In addition to defining a Cartesian plane, the navigation markers of the present invention may be utilized to define positions orthogonal to a plane. For example, three markers can be used to define three points in a given plane. The cross products of vectors in the defined plane can be used to define vectors orthogonal to the plane. Further, multiple navigation markers can be located at identifiable locations referenced to MRI or CT images such that terrain mapping can be performed, again providing for precise registration of locations or objects. Methods of defining and utilizing places, vectors, and points will be readily apparent to those of skill in the art upon reading this disclosure.

Another advantage of using multiple navigation markers is that the relative positions of two or more navigation markers may be tracked in instances where one or more of the markers is moving. For example, a navigation marker in a surgical instrument maybe tracked relative to a fixed navigation marker on the patient's anatomy, such that the relative position between the two is monitored in real time. If the instrument is positioned too close to a particular navigation marker, this fact can be recognized by the tracking system. In addition, in instances wherein the surgical instrument has an active RFID component, and the fixed navigation marker has a passive RFID component, a radio frequency emitted by the active RFID component may serve to generate current within the passive RFID component. In this situation, the two navigation markers are capable of serving as proximity sensors even without the use of a tracking system to monitor the location of the two navigation markers. In some circumstances, the active RFID marker may even transmit to a tracking or other system detailed information about the precise distance between two or more markers. This information may be useful, for example, in the fixation of a broken bone, where proper alignment and joining of broken portions of bone is essential. Separate navigation markers in each portion of broken bone may be used to monitor alignment and positioning of the bone, either actively via RFID or other methods, as described above, or passively through the use of real-time monitoring with a tracking system.

Multiple navigation markers may also be used to define the sterile field of an operative site. In situations wherein navigation markers are also contained within surgical instrumentation, an imaging or tracking system can be used to monitor the location of the surgical instruments and notify a surgeon or operating room staff if a surgical instrument leaves the sterile field. Further, in situations in which the surgical instruments contain active RFID components, these components may at a predefined range induce current in passive RFID components in the navigation markers defining a sterile field such that the passive RFID component of the fixed navigation markers defining the sterile field are only activated if the surgical instruments having the active RFID components move too near the edge of the sterile field.

The navigation markers of the present invention may be constructed from a variety of suitable materials, including stainless steel, titanium alloy, aluminum alloy, and cobalt-chrome alloy. In addition, various synthetic polymers may be employed, either for complete construction of a navigation marker or for coating a navigation marker to impart desired properties to navigation markers constructed from other materials. Any suitable materials may be used while remaining within the spirit and scope of the present invention. Such suitable materials need only have the strength or material properties necessary to the specific embodiment of the navigation marker at issue. For example, in embodiments of the navigation markers that include pins or screws, the marker must be sufficiently strong to be inserted into bone without breaking, and must be able to maintain steady positioning throughout a surgical procedure such that the imaging or tracking system can focus upon the marker. In the embodiments of the navigation markers associated with surgical instrumentation, the navigation markers must meet the necessary material and structural requirements of the surgical instrument. For example, it may be desired that various navigation markers constructed in accordance with the teaching of the present invention be suitable for autoclaving in order to sterilize the marker between use. For navigation markers using RFID or other technology, the material requirements for the navigation markers are such that the RFID or technological component of the marker is adequately protected. Material requirements for a given purpose will be readily ascertained by those of skill in the art upon reading this disclosure.

Although the present invention has been described in its connection with the various embodiments above, those of ordinary skill in the art will understand that many additional modifications can be made to the present invention within the scope of the claims that follow. Accordingly, it is not intended that the scope of the invention be in any way limited by the above description, but be determined entirely by reference to the claims that follow.

What is claimed is:

1. A surgical navigation marker comprising:
a shaft portion having a first and second end, said first end defining a point and said second end defining a head portion of said marker, said head portion comprising a depression defined in a surface thereof, said depression being registrable by an imaging system.

2. The surgical navigation marker according to claim 1 further comprising an opening extending from a surface of said depression into an interior space of the head of said marker, said opening capable of registration by an imaging system.

3. The surgical navigation marker according to claim 1 wherein said shaft portion is a threaded shaft.

4. The surgical navigation marker according to claim 1 wherein said head portion further comprises at least one flattened portion adapted to allow easy manipulation of said surgical navigation marker.

5. The surgical navigation marker according to claim 1 wherein at least a portion of said depression is provided with a color capable of registration by an imaging system.

6. The surgical navigation marker according to claim 1 wherein at least a portion of said depression is provided with scoring capable of registration by an imaging system.

7. The surgical navigation marker according to claim 1 wherein at least a portion of said depression is provided with texturing capable of registration by an imaging system.

8. The surgical navigation marker according to claim 1 further comprising an inversion extending outwardly from a surface of said depression, said inversion capable of registration by an imaging system.

9. The surgical navigation marker according to claim 8 wherein said inversion is provided with a color capable of registration by an imaging system.

10. The surgical navigation marker according to claim 8 wherein said inversion is provided with scoring capable of registration by an imaging system.

11. The surgical navigation marker according to claim 8 wherein said inversion is provided with texturing capable of registration by an imaging system.

12. The surgical navigation marker according to claim 1 further comprising an RFID portion.

13. The surgical navigation marker according to claim 12 wherein said RFID portion is selected from the group consisting of active RFID portions and passive RFID portions.

14. The surgical navigation marker according to claim 1 wherein said surgical navigation marker is fixedly attached to a surgical instrument for registration of a position of said surgical instrument by an imaging system.

15. A method of defining a sterile field during a surgical procedure, the method comprising:
a) providing at least three surgical navigation markers, each of said surgical navigation markers comprising:
a shaft portion having a first and second end, said first end defining a point and said second end defining a head portion of said marker, said head portion comprising a depression defined in a surface thereof, said depression being registrable by an imaging system;
b) positioning each of said at least three surgical navigation markers along an edge of a sterile surgical field;
c) providing an imaging system; and
d) registering the position of each of the at least three surgical navigation markers using said imaging system.

16. The method according to claim 15 further comprising the step of displaying a boundary of said surgical field using said imaging system, said boundary being determined by the registered position of each of the at least three surgical navigation markers.

17. A method of maintaining a sterile field during a surgical procedure, the method comprising:
a) providing a plurality of surgical navigation markers, said surgical navigation markers comprising passive RFID portions;
b) positioning said plurality of surgical navigation markers such that said surgical navigation markers define a border of a sterile field;
c) providing at least one surgical instrument for use during said surgical procedure, said at least one surgical instrument comprising an active RFID portion adapted to induce a current in said passive RFID portions of said surgical navigation markers when said active RFID portion come within a predetermined distance of said passive RFID portions; and
d) providing an alarm in communication with said passive RFID portions of said surgical navigation markers such that said alarm notifies a user of said method when said at least one surgical instrument comes within a predetermined distance of one of said plurality of surgical navigation markers defining said sterile field.

18. A method of calibrating a surgical tracking system comprising:
a) providing a surgical navigation marker at a fixed point within a surgical field′;
b) providing an image of said surgical field in which a surgical procedure is to be performed, said image including a first reference point, a second reference point, and said surgical navigation marker;
c) providing a tracking system for tracking the movement of an object within said surgical field, said tracking system determining the position of said object in relation to said first and second reference points and, based on said relation, superimposing the position of said object on said image;
d) superimposing said surgical navigation marker on said image based on the relation of said surgical navigation marker to said first and second reference points; and
e) calibrating said tracking system by adjusting said tracking system until the superimposed surgical navigation marker aligns with the position of said surgical navigation marker in said image.

19. The method of claim 18 wherein said surgical navigation marker comprises a shaft portion having a first and second end, said first end defining a point and said second end defining a head portion of said marker, said head portion comprising a depression defined in a surface thereof, said depression being registrable by a tracking system.

20. A method of establishing a Cartesian plane within a surgical field comprising:
a) providing a plurality of fixed surgical navigation markers within said field; and
b) establishing a Cartesian plane within said surgical field based upon the relative position of each of said plurality of surgical navigation markers.
